SEQUENCE LISTING (8 sequences)

SEQ ID NO: 1 - protein, 34 aa, artificial
  Derived from Bac7
RRIRPRPRLPRPRPRPLPFPRPGPRPIPRPLPFP

SEQ ID NO: 2 - protein, 39 aa, artificial
  Derived from PR-39
RRRPRPPYLPRPRPPPFFPPRLPPRIPPGFPPRFPPRFP

SEQ ID NO: 3 - protein, 7 aa, artificial
  synthetic PRP based peptide
PRPRPRP

SEQ ID NO: 4 - protein, 7 aa, artificial
  synthetic PRP peptide, all D amino acids
  MISC_FEATURE (1)..(7): D-amino acid
PRPRPRP

SEQ ID NO: 5 - protein, 7 aa, artificial
  synthetic PRP consensus sequence
  VARIANT (4)..(4): R G L or W
PRPZPRP

SEQ ID NO: 6 - protein, 7 aa, artificial
  synthetic PRP variant, G is 4th residue
PRPGPRP

SEQ ID NO: 7 - protein, 7 aa, artificial
  PRP variant 4th residue is L
PRPLPRP

SEQ ID NO: 8 - protein, 7 aa, artificial
  Synthetic PRP peptide, 4th residue is W
PRPWPRP

(19) United States
(12) Patent Application Publication
(10) Pub. No.: US 2020/0407398 A1
(43) Pub. Date: Dec. 31, 2020

(54) Short Proline-Rich Lipopeptide Potentiates Minocycline and Rifampicin Against Multidrug-and Extensively Drug-Resistant Pseudomonas Aeruginosa

(71) Applicant: POLYAMYNA NANOTECH INC., St. John's (CA)

(72) Inventors: Frank Schweizer, Winnipeg (CA); Ronald Domalaon, Winnipeg (CA)

(21) Appl. No.: 16/975,791

(22) PCT Filed: Mar. 8, 2019
(86) PCT No.: PCT/CA2019/050285

§ 371 Date: Aug. 26, 2020

Related U.S. Application Data
(60) Provisional application No. 62/640,318, filed on Mar. 8, 2018.

(51) Int. Cl. C07K 7/06; A61K 31/65; A61K 31/496; A61K 38/08

(52) U.S. Cl. CPC C07K 7/06; A61K 38/08; A61K 31/496; A61K 31/65

(57) ABSTRACT

We evaluated the antibacterial activity of synthetic short proline-rich lipopeptides (SPRLPs) against clinically-relevant Gram-positive and Gram-negative pathogens. The short peptide sequence of SPRLPs were inspired by the repeating PXP motif apparent in longer PRAMPs. We assessed the potential of these SPRLPs to serve as adjuvants in combination with clinically-used antibiotics against P. aeruginosa. Our results revealed an amphiphilic non-hemolytic non-cytotoxic L-lipopeptide lead sequence that strongly potentiates minocycline and rifampicin against MDR/XDR P. aeruginosa. Furthermore, the adjuvant potency is retained in its enantiomeric D-SPRLP counterpart.

### PRIOR APPLICATION INFORMATION

The instant application claims the benefit of U.S. Provisional Patent Application Ser. No. 62/640,318, filed Mar. 8, 2018, entitled “Short proline-rich lipopeptide potentiates minocycline and rifampicin against multidrug- and extensively drug-resistant Pseudomonas aeruginosa”, the entire contents of which are incorporated herein by reference for all purposes.

### BACKGROUND OF THE INVENTION

The increasing incidence of multidrug-resistant (MDR) and extensively drug-resistant (XDR) Pseudomonas aeruginosa infection impose significant burden in our current health care system (1, 2). Infections caused by P. aeruginosa are difficult to treat as this pathogen often harbors multiple resistance mechanisms against most currently-used antibiotics (3, 4). Intrinsic resistance in P. aeruginosa is a major hurdle to overcome. The protective outer membrane (OM) of P. aeruginosa is comprised of selective porins and a polar lipopolysaccharide (LPS) barrier that is 12-100 times less permeable than Escherichia coli (5). Compounds which are able to cross the OM and enter the periplasm are prone to efflux by up to twelve overexpressed multidrug efflux systems that prevent most antibiotics from reaching the required intracellular concentration for their antibacterial action (6, 7). As a result, there is currently a strong interest to identify novel agents that are able to enhance membrane permeability and compromise active efflux in Gram-negative bacillary pathogens such as P. aeruginosa (8).

Proline-rich antimicrobial peptides (PRAMPs) are amphiphilic cationic peptides typically possessing potent Gram-negative but poor Gram-positive antibacterial activity (9, 10). They are characterized by an unusually high amount of L-proline residues (typically 25-50% sequence composition) and frequently contain a repeating PXP or PXXP motif where X may be any amino acid but typically L-arginine (9, 10). Well-known examples include mammalian-derived Bac7(1-35) (11) (sequence: RRIRPRPRLPRPRPRPLPFPRPGPRPIPRPLPFP, SEQ ID No:1) and PR-39 (12) (sequence: RRRPRPPYLPRPRPPPFFPPRLPPRIPPGFPPRFPPRFP-NH₂, SEQ ID No:2), and insect-derived apidaecins (13). PRAMPs eradicate bacteria in a dose-dependent bimodular fashion. At low concentrations, they are believed to target the 70S ribosome and the DnaK chaperone (14, 15). Conversely, they eradicate pathogen via lysis at high concentrations (15). PRAMPs enter the OM via a poorly understood mechanism (presumably through ‘self-promoted’ uptake mechanism similar to most cationic peptides) (16). Inner membrane transporters Sbma and MdtM proteins facilitate their promiscuous cytosolic uptake (17, 18). However, the Gram-negative P. aeruginosa does not express both Sbma and MdtM therefore PRAMPs mode of action is restricted to membrane rupture and lysis (19). With the urgent need for new therapeutic agents/strategies to treat drug-resistant Gram-negative bacterial infections, PRAMPs are considered as an emerging source of potential new antibiotics.

While different from antimicrobials that directly kill bacteria, adjuvants that sensitize resistant pathogens to existing antibiotics are widely studied (20, 21). In fact, several combinations of a β-lactamase inhibitor (adjuvant) and a β-lactam (antibiotic) are already used to treat drug-resistant Gram-negative bacillary infections (22, 23). Adjuvants act on bacterial processes that may elicit direct or indirect advantageous effects towards its partner antibiotic. For instance, adjuvants that inhibit β-lactamase enzymes prevent the degradation of β-lactam antibiotics. Adjuvants that disrupt the bacterial membrane may enhance cellular permeation of otherwise membrane-impermeable antibiotics.

Among infections caused by Gram-negative rods, Pseudomonas aeruginosa has a leading role (43), especially in critically ill and immunocompromised patients. Antimicrobial resistance has led to a serious restriction in treatment options for P. aeruginosa infections, which has become a critical and deadly issue causing a total of 51,000 healthcare infections in the USA per year (44; 45). Despite this problem, physicians mainly rely on retrospective non-randomized controlled studies to derive conclusions about the optimal therapeutic management of these infections.

Eight categories of antibiotics are mainly used to treat P. aeruginosa infections including aminoglycosides (gentamicin, tobramycin, amikacin, netilmicin), carbapenems (imipenem, meropenem), cephalosporins (ceftazidime, cefepime), fluoroquinolones (ciprofloxacin, levofloxacin), penicillin with β-lactamase inhibitors (BLI) (ticarcillin and piperacillin in combination with clavulanic acid or tazobactam), monobactams (aztreonam), fosfomycin and polymyxins (colistin, polymyxin B). Bacteria exhibit multiple resistance mechanisms to antibiotics including decreased permeability, expression of efflux systems, production of antibiotic inactivating enzymes and target modifications. P. aeruginosa exhibits most of these known resistance mechanisms through both intrinsic chromosomally encoded or genetically imported resistance determinants affecting the major classes of antibiotics such as β-lactams, aminoglycosides, quinolones and polymyxins. P. aeruginosa is a pathogen presenting a large genome that can develop a large number of factors associated with antibiotic resistance involving almost all classes of antibiotics. The strains of P. aeruginosa are categorized as follows: (1) MDR when resistance is observed in ≥1 agent in ≥3 categories; (2) extensively drug-resistant (XDR) when a resistance is observed in ≥agent in all but ≤categories; and (3) pandrug-resistant (PDR) when the strain is non-susceptible to all antimicrobial agents.

Role of combination therapy in P. aeruginosa infections:

The 2 main reasons quoted in favor of combination therapy for P. aeruginosa are (a) to increase the probability of appropriate empirical coverage and (b) to improve overall the antibiotics' activity through synergism (46).

Early administration of an adequate antibiotic regimen has been associated with favorable clinical outcome, especially among critically ill patients presenting with severe P. aeruginosa infections (47). Conversely, a delay in the prescription of an adequate antibiotic therapy has been related to a significant increase in mortality.

In recent years, the progressive increase in antibiotic resistance among P. aeruginosa has been identified as the main reason for antibiotic inadequacy, with a negative impact on patient survival (48). The available evidence suggests that the greatest benefit of a combination therapy stems from the increased likelihood of choosing an effective agent during empirical therapy rather than to prevent the resistance during definitive therapy or to benefit of in vitro synergistic activity. Therefore, to balance between early antibiotic administration and risk of resistance selection, physicians suggest early administration of a combination regimen when P. aeruginosa is suspected, followed by a prompt de-escalation when the antimicrobial susceptibility testing becomes available. Generally, physicians encourage an approach consisting of the prescription of an anti-pseudomonal β-lactam (piperacillin/tazobactam, ceftolozane/tazobactam, ceftazidime, cefepime, or a carbapenem) plus a second anti-pseudomonal agent (aminoglycoside or a fluoroquinolones).

The difficulty in treating P. aeruginosa infections caused by strains that are resistant to all or all but one antibiotic has led investigators to use novel combinations of drugs that separately have little or no activity against the isolate (49; 50). There are minimal clinical data to support such combination therapy, and most clinical studies did not analyze patient outcomes stratified by susceptibility profile. However, if combination therapy is used for treatment of organisms with extreme or unusual multidrug resistance patterns, it should be done in consultation with an expert in treating such infections whenever possible.

In a single clinical series of 64 patients with nosocomial pulmonary infections due to a highly resistant P. aeruginosa susceptible only to colistin, treatment with the combination of cefepime and amikacin was associated with survival in 44 (69 percent) (51). These agents were the least inactive antibiotics by MIC determination and had demonstrated synergy in vitro.

### SUMMARY OF THE INVENTION

According to a first aspect of the invention, there is provided a compound comprising the peptide as set forth in SEQ ID No:3 or SEQ ID No:4 connected to a C9-C13 aliphatic chain. According to another aspect of the invention, there is provided a method of treating a bacterial infection comprising: co-administering an effective amount of a compound as described above and an effective amount of a suitable antibiotic to an individual in need of such treatment. According to another aspect of the invention, there is provided a method of permeabilizing a Gram-negative bacterial membrane comprising: administering an effective amount of the compound as described above to a suitable Gram-negative bacterium.

According to another aspect of the invention, there is provided use of the compound as described above for treating a Gram-negative bacteria infection.

### BRIEF DESCRIPTION OF THE DRAWINGS

FIG. 1. Evaluation for cytotoxicity of amphiphilic C12-PRP (red circle) via (A) hemolytic activity against erythrocytes, (B) inhibition of cellular proliferation against human liver carcinoma HepG2 cells, (C) inhibition of cellular proliferation against human embryonic kidney HEK-293 cells, (D) cytotoxic effects against human liver carcinoma HepG2 cells and (E) cytotoxic effects against human embryonic kidney HEK-293 cells. All experiments were performed in three or more replicates. Colistin (blue square) and adriamycin® (green triangle) were used to represent clinically-used peptide antibiotics and anticancer drugs, respectively. Error bars indicate standard deviation from three independent experiments (n=3).

FIG. 2. Exemplary examples of suitable C9-C13 aliphatic compounds.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference for all purposes.

As used herein, an “adjuvant” refers to a compound that increases the sensitivity of bacteria to an antibiotic. As will be apparent to one of skill in the art, this can be measured by a variety of ways known in the art, for example, by a decreased minimum inhibitory concentration of an antibiotic of interest when the adjuvant is administered with the antibiotic of interest compared to the antibiotic of interest administered alone, or a fractional inhibitory concentration of less than 0.5, as discussed herein. It is of note that the adjuvant compound can be co-administered with the antibiotic of interest by administering both compounds together, or by administering one compound prior to the other. As will be apparent to one of skill in the art, as used herein, “co-administered” refers to the fact that the compound of the invention and the suitable antibiotic are administered such that both are biologically active at the same time or for an overlapping period of time.

Herein, we evaluate the antibacterial activity of synthetic short proline-rich lipopeptides (SPRLPs) against clinically-relevant Gram-positive and Gram-negative pathogens. The short peptide sequence of SPRLPs were inspired by the repeating PXP motif apparent in longer PRAMPs. Moreover, we assess the potential of these SPRLPs to serve as adjuvants in combination with clinically-used antibiotics against P. aeruginosa. Our results revealed an amphiphilic non-hemolytic non-cytotoxic L-lipopeptide lead sequence that strongly potentiates minocycline and rifampicin against MDR/XDR P. aeruginosa. Furthermore, the adjuvant potency is retained in its enantiomeric D-SPRLP counterpart.

As discussed herein, a series of 16 short proline-rich lipopeptides (SPRLPs) were constructed to mimic longer naturally-existing proline-rich antimicrobial peptides. Antibacterial assessment revealed that lipopeptides containing hexadecanoic acid (C16) possess optimal antibacterial activity relative to others with a shorter lipid component. SPRLPs were further evaluated for their potential to serve as adjuvants in combination with existing antibiotics to enhance antibacterial activity against drug-resistant Pseudomonas aeruginosa.

As will be appreciated by one of skill in the art and as discussed herein, P. aeruginosa infections are very difficult to treat because of the drug resistance associated with the bacterium. Specifically, as discussed herein, Pseudomonas aeruginosa is an emergent pathogen with high intrinsic antibiotic resistance and is among the most common hospital pathogens (15). Serious P. aeruginosa infections are often associated with compromised host defenses such as in neutropenia, severe burns, or cystic fibrosis (CF) (16). These infections demonstrate high morbidity and mortality for the limited therapies, in particular due to the spread of antimicrobial-resistant strains (17). Furthermore, as discussed herein, this bacterial strain has a number of different mechanisms for antibiotic resistance, meaning that this bacterium serves as an excellent model system for all Gram-negative bacteria.

Out of sixteen prepared SPRLPs, C12-PRP was found to significantly potentiate the antibiotics minocycline and rifampicin against multidrug- and extensively drug-resistant (MDR/XDR) P. aeruginosa clinical isolates. This non-hemolytic C12-PRP is comprised of a heptapeptide sequence PRPRPRP-NH₂ (SEQ ID No:3) acylated to dodecanoic acid (C12) at the N-terminus. The adjuvant potency of C12-PRP was apparent by its ability to reduce the minimum inhibitory concentration of minocycline and rifampicin below their interpretative susceptibility breakpoints against MDR/XDR P. aeruginosa. An attempt to optimize C12-PRP through peptidomimetic modification was performed by replacing all L- to D-amino acids (SEQ ID No:4). C12-PRP demonstrated pliability to optimization as synergism with minocycline and rifampicin were retained. Moreover, C12-PRP displayed no cytotoxicity against human liver carcinoma HepG2 and human embryonic kidney HEK-293 cell lines. Discovery of agents that are able to resuscitate activity of existing antibiotics against drug-resistant Gram-negative pathogens, especially P. aeruginosa are of great clinical interest.

An amphiphilic short proline-rich lipopeptide that synergizes with two clinically-used antibiotics was identified for the first time. The non-hemolytic lipopeptide C12-PRP, with a short sequence of C12-PRPRPRP-NH₂ (SEQ ID No:3), potentiates minocycline and rifampicin against wild-type and multidrug-/extensively drug-resistant P. aeruginosa. More importantly, C12-PRP significantly reduced the MIC of minocycline and rifampicin against P. aeruginosa below their interpretative susceptibility breakpoints, as discussed below. Furthermore, our data indicates that C12-PRP is non-cytotoxic. Our initial attempt of optimization by incorporating D-amino acids retained the desired adjuvant property of the lipopeptide; and therefore C12-PRP appeared to be amenable to peptidomimetic modification.

Indeed, peptide-based antibacterial drug candidates such as murepavadin (also known as POL7080) (35) and brilacidin (36), both in Phase-2 clinical trials, were optimized to remove their ‘peptide-like’ nature prior to clinical validation. Furthermore, the in vivo efficacy of the lipopeptide+antibiotic combination will be assessed in insect models of infection. Mode of action studies to explore the effects of the lead lipopeptide on the OM, inner membrane and proton motive force that can result in increased intracellular concentrations of minocycline and rifampicin are planned (39, 40).

We evaluated the activity of SPRLPs in combination with fifteen clinically-used antibiotics against P. aeruginosa. Antibiotics tested included fluoroquinolones (moxifloxacin, ciprofloxacin and levofloxacin), aminoglycosides (gentamicin, tobramycin and amikacin), cephalosporins (ceftazidime and cefotaxime), carbapenems (meropenem and doripenem), aztreonam, rifampicin, minocycline, colistin and fosfomycin. The SPRLPs were screened initially at a fixed concentration of 8 μg/mL (5 μM) in combination with antibiotics against wild-type P. aeruginosa PAO1. We assessed potentiation by at least a four-fold absolute MIC reduction of the antibiotic, after which synergism was further validated by a conventional checkerboard assay. A fractional inhibitory concentration (FIC) index of ≤0.5, 0.5<x≤4 or >4 was interpreted as synergistic, indifferent or antagonistic interaction, respectively (31). FIC index was obtained by adding the FIC values of the antibiotic and the SPRLP adjuvant. FIC of antibiotics was calculated by dividing the MIC of the antibiotic in the presence of the adjuvant by the MIC of the antibiotic alone. Similarly, the FIC of adjuvant was calculated via dividing the MIC of the adjuvant in the presence of the antibiotic by the MIC of the adjuvant alone.

Out of the fifteen clinically-used antibiotics and sixteen short synthetic SPRLPs, initial screening revealed potentiation of minocycline and rifampicin with the amphiphilic lipopeptide C12-PRP. Further validation by checkerboard assay confirmed the synergistic combinations against wild-type P. aeruginosa PAO1 strain. C12-PRP with either minocycline or rifampicin yielded an FIC index of 0.19 or 0.14, respectively. These findings warranted further studies as C12-PRP is non-hemolytic even up a high concentration of 512 μg/mL. We evaluated whether the observed synergism was retained against MDR/XDR P. aeruginosa clinical isolates. Also, the capability of C12-PRP to reduce the absolute MIC of minocycline and rifampicin below their susceptibility breakpoint was investigated. No established minocycline and rifampicin susceptibility breakpoints currently exist for Pseudomonas aeruginosa from neither Clinical and Laboratory Standards Institute (CLSI) nor the European Committee on Antimicrobial Susceptibility Testing (EUCAST). Therefore, we cautiously used established breakpoints for other organisms as similar as possible to Pseudomonas aeruginosa for our comparison According to CLSI (32), the susceptibility breakpoint of minocycline for Acinetobacter spp. is ≤4 μg/mL while the susceptibility breakpoint of rifampicin for Staphylococcus spp. is ≤1 μg/mL.

The purpose of undertaking susceptibility testing, by whatever method, is to attempt to integrate the drug potency against a population of potential pathogens with the pharmacokinetics of the antimicrobial and, whenever possible, to review this relationship in the light of clinical experience following therapy in clinical trials. Breakpoints are discriminatory antimicrobial concentrations used in the interpretation of results of susceptibility testing to define isolates as susceptible (S), intermediate or resistant (R). Clinical, pharmacological, microbiological and pharmacodynamic considerations are important in setting breakpoints. An antimicrobial breakpoint is the agreed concentration of an agent at which bacteria can, and cannot, be treated with the antimicrobial agent in question. This will be related to the dose needed to treat susceptible bacteria. Essentially, the breakpoint is a man-made or rather decided concentration, which corresponds to a dose required to inhibit bacterial growth in relevant infections.

The combination of minocycline and C12-PRP was found to be strongly synergistic against all eight tested MDR/XDR P. aeruginosa isolates (Table 4). Moreover, the MIC of minocycline in the presence of 8 μg/mL (5 μM) C12-PRP was reduced below susceptibility breakpoint in seven out of nine strains.

The process by which breakpoints are defined is based on the work of a committee formed by individuals from different backgrounds in clinical microbiology and infectious diseases. After research into and discussions of parameters—such as antimicrobial activity, resistance mechanisms, pharmacokinetics, and pharmacodynamics—clinical outcome data will define the concentration which will serve as the breakpoint. At the end of the process, it is published as a tentative breakpoint on the CLSI or EUCAST website and opened for consultation. Opinions are gathered and discussed, and final breakpoints and rebuttals are eventually published. However, neither agency reported or published susceptibility breakpoint values for P. aeruginosa (for minocycline and rifampicin). Hence, we relied on other bacteria with known breakpoint values, which possess similar structural/resistance patterns (reason for choosing A. baumannii) or other characteristics such as treatment methods (reason for choosing Staphylococcus) to interpret breakpoint values for P. aeruginosa.

In case one, to interpret breakpoint values for minocycline we opted for another Gram-negative organism i.e., A. baumannii, which also exert high levels of intrinsic mechanism and infections associated with A. baumannii are being treated with minocycline.

Minocycline is a second-generation, semisynthetic tetracycline derivative that was first introduced in the 1960s. While the oral formulation remained available, the intravenous formulation was taken off the US market in 2005 due to decreased use. It was re-introduced in 2009 and has become an important option for the treatment of multidrug-resistant organisms, in particular carbapenem-resistant A. baumannii (CRAB). Following reintroduction of minocycline to the market for the treatment of serious infections, including an FDA-approved indication for infections caused by Acinetobacter, there has been renewed interest in its use. Further investigation of its use for the treatment of MDR Acinetobacter infections, including those caused by carbapenem-resistant and XDR strains, has occurred due to its in vitro activity against A. baumannii, more favorable pharmacokinetics compared to tigecycline, and its safety profile. With the recent reintroduction of an intravenous formulation of minocycline, there is increasing interest in this agent as an additional treatment option for carbapenem-resistant A. baumannii infections. In the aforementioned global survey, over 70% of the clinical isolates were susceptible to minocycline, using the CLSI breakpoints of ≤4 μg/ml for susceptibility, 8 μg/ml for intermediate resistance, and ≥16 μg/ml for resistance.

Minocycline has been shown to overcome many resistance mechanisms affecting other tetracyclines in A. baumannii, including tigecycline. Additionally, it has favorable pharmacokinetic and pharmacodynamic properties, as well as excellent in vitro activity against drug-resistant A. baumannii. Therefore, we envisioned, if we could somehow bring minocycline breakpoint level to ≤4 μg/ml, then that particular “combination regimen” would be considered clinically significant for multi-drug resistant P. aeruginosa as well. In this study, we have achieved the susceptible breakpoint values for majority of the multidrug resistant P. aeruginosa strains.

In case two, to predict breakpoints for rifampicin (against P. aeruginosa) we have chosen Staphylococcus, since rifampicin is particularly being used to treat Gram-positive pathogens. The idea is to prompt inefficient antibiotics (against Gram-negative bacteria) to improve activity against Gram-negative P. aeruginosa. The potential usefulness of these combinations is to provide future therapeutic alternatives for Gram-negative bacterial infections. Rifampin is a hydrophobic antibiotic, and targets cytoplasmic and periplasmic bacterial components. Rifampin's inefficacy over Gram-negative bacteria usually results from its natural low outer membrane -permeability, although other mechanisms were reported to confer additional resistance to rifampin, including mutations in RNA polymerase gene, rpoB. Rifampin is active on Gram-positive cocci but not on enterobacteriaceae- a large family of Gram-negative bacteria that includes common pathogens, such as E. coli, Klebsiella, Salmonella or Pseudomonas. Clearly, the opportunity to expand rifampin's activity spectrum and/or to reduce its adverse effects (e.g., hepatotoxicity), would be welcomed. Hence, we tried to potentiate rifampicin with C12-PRP. About half of the MDR strains reached susceptibility breakpoints (≤1 μg/mL) if we consider Staphylococcus breakpoints. However, most of the MDR strains reached susceptibility levels (is ≤4 μg/mL) if we consider A. baumannii as standard. Furthermore, significant potentiation was also observed for the combination of rifampicin and C12-PRP against MDR/XDR P. aeruginosa isolates (Table 5). At 8 μg/mL (5 μM) C12-PRP, the MIC of rifampicin reached susceptibility breakpoint in five out of nine strains. Indeed, the SPRLP C12-PRP was able to enhance the antibacterial potency of minocycline and rifampicin against wild-type and clinical isolates of P. aeruginosa. While not wishing to be bound to a particular theory or hypothesis, the inventors believe that membrane perturbation that results in enhanced antibiotic uptake is a likely possibility. PRAMPs are known to disrupt bacterial membranes, which is more pronounced in P. aeruginosa relative to other Gram-negative bacilli (19). The SPRLP C12-PRP may potentiate minocycline and rifampicin through OM permeabilization of P. aeruginosa. Membrane perturbation may also compromise the activity of integral membrane proteins such as multidrug efflux pumps, essentially halting antibiotic resistance through active efflux.

The fatty acyl ligated to the peptide sequence PRPRPRP-NH₂ matters for the adjuvant property, as discussed herein. Since the lead adjuvant C12-PRP was discovered from a synergy scan having a fixed 8 μg/mL (5 μM) SPRLP concentration, combinations of minocycline or rifampicin with other PRP subset lipopeptides warranted further investigation. Therefore, we assessed the interaction of either C8-PRP, C16-PRP or Ad-PRP with minocycline or rifampicin by checkerboard assay. The three synthetic SPRLPs displayed indifferent interaction with minocycline and rifampicin. Interestingly, C16-PRP did not display synergism with either antibiotic even though our initial data suggested that it can disrupt and lyse membranes. These data suggest that the aliphatic lipid C12 is optimal for amphiphilic SPRLPs to potentiate minocycline and rifampicin against P. aeruginosa. According to an aspect of the invention, there is provided a compound comprising the peptide as set forth in SEQ ID No:3 or SEQ ID No:4 connected to a C9-C13 aliphatic chain.

According to an aspect of the invention, there is provided a compound comprising the peptide as set forth in SEQ ID No:3 or SEQ ID No:4 connected to a C9-C13 aliphatic chain for treating a bacterial infection.

According to an aspect of the invention, there is provided a compound comprising the peptide as set forth in SEQ ID No:3 or SEQ ID No:4 connected to a C9-C13 aliphatic chain for increasing membrane permeability of a Gram-negative bacterium.

According to an aspect of the invention, there is provided a compound comprising the peptide as set forth in SEQ ID No:3 or SEQ ID No:4 for treating a bacterial infection in combination with a suitable antibiotic.

As will be apparent to those of skill in the art, an aliphatic chain or aliphatic compound is a hydrocarbon compound containing carbon and hydrogen joined together in straight chains, branched trains or non-aromatic rings. Aliphatic compounds may be saturated (e.g., hexane and other alkanes) or unsaturated (e.g., hexene and other alkenes, as well as alkynes). Alternatively, the aliphatic chain or compound may be referred to as a lipophilic moiety or as a lipophilic compound. As will be appreciated by one of skill in the art, the aliphatic chains are essentially hydrophobic scaffolds.

As will be apparent to one of skill in the art and as discussed herein, “C9-C13 aliphatic chain” does not necessarily need to be “composed” of 9-13 carbons, but needs to be of a similar length as a chain of 9-13 carbons.

In some embodiments, the C9-C13 aliphatic chain is about 9 to about 13 carbon atoms long or about 9 to about 12 carbon atoms long or about 10 to about 13 carbon atoms long or about 10 to about 12 carbon atoms long or about 11 to about 13 carbon atoms long or about 12 to about 13 carbon atoms long or about 11 to about 12 carbon atoms long or about 12 carbon atoms long. As used herein, “about” refers to a value that is within “about” 10% of the recited value. As discussed herein, in some embodiments, the peptide is connected to the C9-C13 aliphatic chain at the N-terminus of the peptide. In other embodiments, the C9-C13 aliphatic chain may be connected to the peptide at the C-terminus of the peptide.

In some embodiments, the peptide is connected to the C9-C13 aliphatic chain by acylation, although other suitable methods may be used within the invention. As will be apparent to one of skill in the art, the exact means used will of course depend on the chemical groups available on the C9-C13 aliphatic chain for linkage, but alternatives to acylation will be readily apparent to one of skill in the art.

As will be appreciated by one of skill in the art, any aliphatic chain that is hydrophobic and/or lipophilic that has a length of about 9 to 13 carbon atoms, for example, about 9 to about 12 carbon atoms, about 10 to about 13 carbon atoms, about 10 to about 12 carbon atoms, about 11 to about 13 carbon atoms, about 12 to about 13 carbon atoms, about 11-12 carbon atoms or about 12 carbon atoms, may be used within the invention.

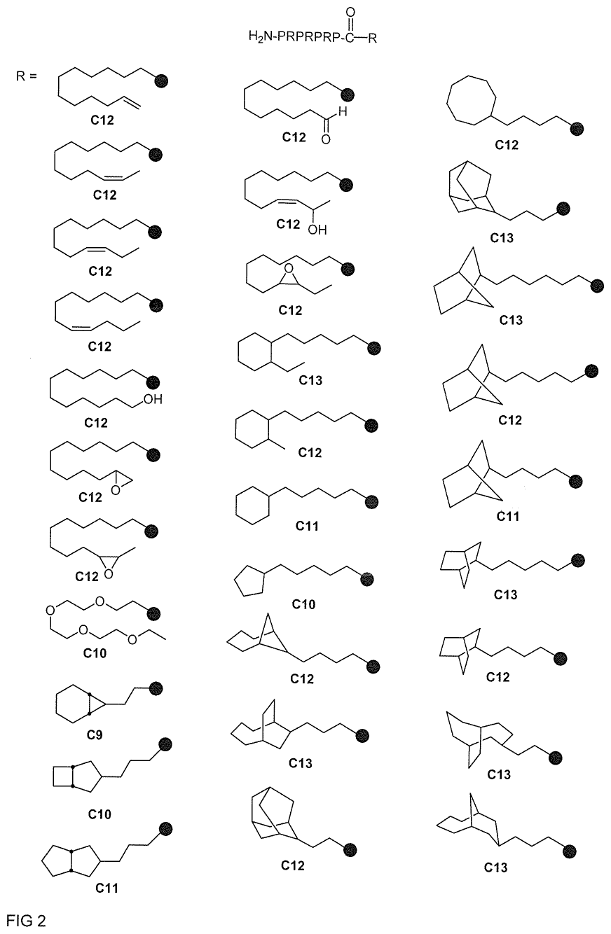

Examples of suitable C9-C13 aliphatic compounds include but are by no means limited to unsubstituted and suitably substituted compounds such as dodecanoic acid, tridecanoic acid, undecanoic acid, decanoic acid and nonanoic acid, as well as other compounds with long aliphatic chains, such as for example but by no means limited to (E)-2-octenal, (E)-2-nonenal, (E)-2-decenal, and (E,E)-2,4-decadienal. As will be appreciated by one of skill in the art, a “suitable substituent” is one that will not change the aliphatic character of the compound. Additional exemplary examples of some suitable C9-C13 aliphatic chains or compounds can be seen in FIG. 2. It is noted that other suitable C9-C13 aliphatic compounds will be readily apparent to one of skill in the art in view of the teachings herein and in the prior art.

As will be known by those of skill in the art, the general pharmacophore determining antibacterial activity in cationic peptides appears to be the presence of protonatable positive charges and hydrophobic groups, i.e., cationic amphipathicity. Recently it has been reported that the order of the amino acids in the peptides was less important for antibacterial activity than the net content of bulky and lipophilic groups. However, the shorter peptides with the bulky and lipophilic groups nearby tended to be slightly more active (53). Thus, we believe that the lipophilic part plays a pivotal role in the antibacterial property of PRAMPs. As discussed herein, a C-9 to C-13 carbon chain length, for example, a C-12 carbon chain length provides optimum antibacterial activity and/or contributes to the synergy effect when administered with suitable antibiotics as discussed herein. As discussed herein, as the carbon number increases, so does the cytotoxicity; however, a C-12 aliphatic containing PRAMPs does not exert significant hemolysis and cytotoxicity.

It is further noted that there have been many powerful antimicrobial agents isolated from plants. Among the active compounds characterized against pathogenic bacteria are aliphatic alcohols. For example, linalool, nerolidol, geraniol, 1-octanol and α-terpineol are among those whose antimicrobial activity has been reported. Accordingly, in some embodiments of the invention, C9-C13 aliphatic chain is a C9-C13 aliphatic chain with active hydrophobic tails. For example, in these embodiments, the C9-C13 aliphatic chain may be 1-heptanol, 2-undecanol, 3-tridecanol, geraniol, farnesol, linalool, nerolidol or geranilacetol.

According to another aspect of the invention, there is provided a method of treating a bacterial infection comprising: co-administering an effective amount of a compound described above and an effective amount of a suitable antibiotic to an individual in need of such treatment.

As will be apparent to one of skill in the art and as discussed herein, the selection of a “suitable” antibiotic will depend on the bacterium known to be or suspected of being responsible for the infection. However, as discussed herein, because of the adjuvant activity of the compounds of the invention, the suitable antibiotic does not necessarily need to be an antibiotic known to have activity of efficacy against the infecting bacterium on its own.

The antibiotic may be selected from the group consisting of: a cephalosporin; a lincosamide; a monobactam; a nitrofuran; an oxazolidone; a penicillin; a penicillin combination; a quinolone; a sulfonamide; a tetracycline; or another suitable antibiotic class.

It is of note that suitable specific antibiotics within these categories will be readily apparent to one of skill in the art. Examples of suitable members of each class are however provided for illustrative purposes.

The cephalosporin may be selected from the group consisting of: cefaclor; cefoxitin; cefoteton; cefamandole; cefinetazole; cefonicid; loracarbef; cefprozil; cefuroxime; cefixime; cefdinir; cefoperazone; cefotaxime; cefpodoxime; ceftazidime; ceftibuten; ceftozoxime; latamoxef; ceftriaxone; cefepime; and ceftobiprole.

The lincosamide may be selected from the group consisting of clinadamycin; and lincomycin.

The monobactam may be for example aztreonam.

The nitrofuran may be for example furazolidone or nitrofurantoin.

The oxazolidone may be for example linezolid or torezolid.

The penicillin may be for example amoxicillin or flucloxacillin.

The penicillin combination may be selected from the group consisting of: amoxixillin/clavulanate; ampicillin/sulbactam; piperacillin/tazobactam; and ticarcillin/clavulanate

The quinolone may be selected from the group consisting of: ciprofloxacin; enoxacin; gatifloxacin; gemifloxacin; levofloxacin; lomefloxacin; moxifloxacin; nadifloxacin; norfloxacin; and ofloxacin.

The sulfonamide may be selected from the group consisting of: mafenide; sulfacetamide; sulfadiazine; silver sulfadiazine; sulfadimethoxine; sulfamethizole; sulfamethoxazole; sulfanilamide; sulfasalazine; sulfisoxazole; trimethoprim-sulfamethoxazole; and sulfonamidochrysoidine.

The tetracycline may be selected from the group consisting of demeclocycline; doxycycline; metacycline; minocycline; oxytetracycline; tetracycline; and tigecycline.

Other suitable antibiotics include but are by no means limited to: clofazimine; dapsone; capreomycin; cycloserine; isoniazid; pyrazinamide; rifampicin; ritabutin; rifapentine; arsphenamine; Fosfomycin; mupirocin; trimethoprim; fidaxomicin; doripenem; ceftaroline; tedizolid; dalbavancin; bedaquiline; quinupristin/dalfopristin; ceftolozane/tazobactam; ceftazidime/avibactam; and daptomycin

The individual in need of such treatment may be an individual who is suspected of having a bacterial infection or who has been diagnosed with a bacterial infection.

The bacterial infection may be suspected of being caused by or known to be caused by a drug-resistant bacterial strain. In some embodiments, the bacterial strain is a Gram-negative bacteria or bacterium.

In some embodiments of the invention, the compound or composition of the invention and the suitable antibiotic are administered as a “fixed-dose combination” (FDC) that includes two active pharmaceutical ingredients (APIs) combined in a single dosage form, which would be manufactured and distributed in fixed doses. For example, the World Health Organization recommends prescribers use fixed-dose combinations to reduce the number of tablets that people take.

As will be apparent to those of skill in the art, the time limit between administrations is based on the severity of the infection, although usual time intervals typically range between 8-12 hours. As will be appreciated by one of skill in the art, the period of time or dosage regimen during which the individual in need of such treatment is administered or takes the combination may depend on the severity of the invention but may be for example at least 5 days or at least one week.

According to another aspect of the invention, there is provided a method of permeabilizing or increasing the permeability of a membrane of a Gram-negative bacterium comprising: administering an effective amount of the compound or composition as described above to a suitable Gram-negative bacterium. As will be apparent to one of skill in the art, the compound may be administered in vivo or ex vivo.

While not wishing to be bound to a particular theory or hypothesis, it has been postulated that membrane-active antimicrobial peptides (AMPs) selectively disrupt the cell membrane to form pores that allow efflux of essential ions or nutrients (54). Based on the Shai-Matsuzaki-Huang (SMH) model, it is believed that most AMPs act via an interaction with the membrane resulting in a morphological change of membrane structure (55).

PRAMPs generally penetrate the outer membrane of Gram-negative bacteria and translocate into the cytoplasm via a permease/transporter-mediated uptake. (56). Accordingly, the compound or composition of the invention can be used to permeabilize or increase permeability of a membrane of any suitable Gram-negative bacteria. As will be apparent to one of skill in the art and as discussed herein, the treated membrane is more permeable than an untreated control bacterial membrane from a similar bacterium. It is of note that a wide variety of means for measuring permeability of bacterial membranes are well known to those of skill in the art and may be used within the invention.

As will be appreciated by one of skill in the art, the compound or composition of the invention may be used to increase permeability of any suitable Gram-negative bacterium. Accordingly, the compound or composition of the invention may be used with anything that would benefit from increased bacterial membrane permeability.

In another aspect of the invention, there is provided use of the compound or composition of the invention for treating a bacterial infection. The bacteria may be a Gram-negative bacterium. The bacteria causing the bacterial infection may be a drug-resistant bacterium.

The individual in need of such treatment is an individual who is suffering from or who is suspected of having a bacterial infection.

In some embodiments, the bacterial infection is caused by a bacterial strain that is suspected of being drug-resistant, or a bacterial strain that has been identified as a drug-resistant bacterial strain

As discussed herein, the compound of the invention may be administered and/or may be formulated to be administered in any suitable form known in the art, for example but by no means limited to an oral dosage form, an injectable dosage form, a transdermal dosage form, an inhalation dosage form, a topical dosage form, and a rectal dosage form.

That is, the compound of the invention may be prepared or formulated to be administered in a variety of ways, for example, topically, orally, intravenously, intramuscularly, subcutaneously, intraperitoneally, intranasally or by local or system intravascular infusion using means known in the art.

Aspects of the invention include dosage forms, formulations, compositions and/or devices containing a PRAMPs as adjuvant and/or agent that primarily increase the permeability of a Gram-negative bacterial cell membrane. The present invention includes, for example, doses and dosage forms for at least oral administration, transdermal delivery, topical application, wound dressings, coating material, suppository delivery, transmucosal delivery, injection (including subcutaneous administration, subdermal administration, intramuscular administration, depot administration, and intravenous administration, including delivery via bolus, slow intravenous injection, and intravenous drip), infusion devices (including implantable infusion devices, both active and passive), administration by inhalation or insufflation, buccal administration and sublingual administration.

In some embodiments, there is provided a pharmaceutical composition comprising the compound of the invention and a suitable and/or pharmaceutically acceptable carrier or excipient.

Suitable carriers and excipients, for example, pharmaceutically suitable excipients and carriers, are well known in the art and suitable examples will of course be readily apparent to one of skill in the art.

In these embodiments, the compound may be formulated to be administered and/or administered in an effective amount, for example, in a pharmaceutically effective amount. As will be apparent to one of skill in the art, a “therapeutically effective amount” or an “effective amount” is an amount of the compound that is sufficient to alleviate or reduce the severity of or treat at least one symptom associated with the disease or condition, in this case, a bacterial infection. As discussed herein, in some embodiments, the effective amount may be determined from the calculated minimum inhibitory concentration, that is, be administered or formulated for administration at an amount that will result in a concentration above, preferably 2-10×, the MIC at the site of the bacterial infection.

As will be apparent to one of skill in the art, the type of symptoms will depend on the bacterial strain causing or believed to be causing or substantially responsible for the infection and will also depend on the location of the infection.

As will be appreciated by one of skill in the art, the specific effective dose may depend on several factors, including but by no means limited to the type of infection, the severity of the infection, the age and/or overall condition and/or weight and/or general health of the patient, the severity of symptoms, the route of administration and the duration of treatment. In some embodiments, the dosage may start at low levels and be gradually increased until the desired effect is achieved.

The invention will now be further explained and/or elucidated by way of examples. However, the invention is not necessarily limited to the examples.

### EXAMPLE 1—D-LIPOPEPTIDE COUNTERPART OF C12-PRP RETAINS ADJUVANT POTENCY

The amphiphilic C12-PRP is considered susceptible to non-specific proteolytic degradation as host enzymes (e.g. human proteases) easily recognize L-amino acid peptide bonds. Therefore, lead peptide agents typically undergo peptidomimetic modifications to increase serum stability (10, 33). We explored one approach to optimize C12-PRP (SEQ ID No:3) by synthesizing the same sequence but with D- instead of L-amino acids, yielding the D-lipopeptide analog C12-prp (SEQ ID No:4). Peptide bonds formed by D-amino acids are less prone to mammalian proteases (34). Similar to C12-PRP (SEQ ID No:3), C12-prp (SEQ ID No:4) was found to be inactive (MIC>128 μg/mL) against wild-type and clinical isolates of P. aeruginosa. The adjuvant properties of C12-PRP were retained but the potency was slightly reduced in the D-lipopeptide analog. C12-prp potentiated minocycline and rifampicin against wild-type and MDR/XDR P. aeruginosa (Tables 6 & 7). Furthermore, 8 μg/mL (5 μM) of C12-prp reduced the MIC of minocycline (Table 6) and rifampicin (Table 7) below susceptibility breakpoints in some MDR/XDR P. aeruginosa clinical isolates. These results suggest that C12-PRP is pliable to peptidomimetic alterations and that further lead optimizations are possible.

Amphiphilic C12-PRP is not cytotoxic to eukaryotic cells. Our initial assessment on the effect of SPRLPs to eukaryotic membranes revealed that C12-PRP is non-hemolytic. In fact, the concentration to result in 5% red blood cell hemolysis for C12-PRP was >512 μg/mL.

The hemolytic activity is an important indicator for cytotoxicity. The low hemolytic activity of the proline-rich peptides provides evidence that the membrane effects are specific to bacteria and not to eukaryotic cells. Several of the antimicrobial peptides are toxic at concentrations necessary for treatment of severe infections, which evidences the importance of knowing and determining both action mechanisms and structural characteristics that influence toxicity. Although the action mechanism of AMPs on the cell have not been elucidated, it is believed that these compounds interact with polar lipid heads from bacterial membranes causing different effects, such as: 1) pore formation, 2) membrane lysis, 3) formation of lipid-protein domains, 4) induction of non-laminar phases, and 5) disintegration among negatively charged lipids from zwitterionic ones.

C12-PRP selectively targets bacterial membranes and not the eukaryotic cells (as evident by low hemolytic activity (4.6±0.2% hemolysis even at 512 μg/mL concentration).

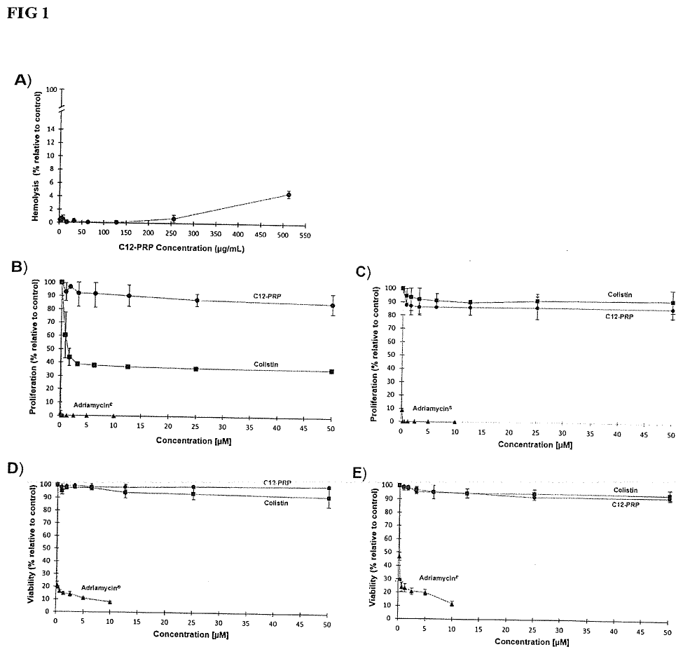

At 512 μg/mL (366 μM), C12-PRP only resulted in 4.6±0.2% hemolysis (FIG. 1A). We then evaluated the potential toxicity of C12-PRP against two eukaryotic cell lines, human liver carcinoma HepG2 and human embryonic kidney HEK-293, by its ability to inhibit cellular proliferation and cellular viability. We used colistin (also known as polymyxin E) and adriamycin® as internal controls to represent clinically-used peptide antibiotics and anticancer drugs, respectively. Amphiphilic C12-PRP did not inhibit cellular proliferation of either cell line (FIGS. 1B and 1C) up to the highest concentration tested (50 μM), notably 10-fold higher than the C12-PRP's adjuvant working concentration (5 μM). Interestingly, 1.5 μM of the antibiotic colistin inhibited proliferation of HepG2 cells to 50% (IC50). The anticancer drug adriamycin® inhibited growth of both cell lines at very low concentrations. We further evaluated cytotoxicity by assessing the effect of the agents on the global oxidoreductive metabolism of cells through MTS assay (FIGS. 1D and 1E). Both C12-PRP and colistin did not kill both cell lines up to the highest concentration tested (50 μM). Congruent with results from the proliferation assay, adriamycin® drastically reduced viability of both cell lines at low concentration. Our data presented herein indicates that the amphiphilic C12-PRP is not cytotoxic to eukaryotic cells.

As discussed herein, only the C12 containing compound displays synergism. As discussed above, the more carbons added, the greater the degree of hemolysis and cytotoxicity observed.

### EXAMPLE 2—SPRLP DESIGN INSPIRED BY REPEATING PXP MOTIF IN PRAMPs

Inspired by peptide sequences of longer and naturally occurring PRAMPs such as Bac7(1-35) (SEQ ID No:1) and PR-39 (SEQ ID No:2), we prepared shorter synthetic versions possessing a lipoheptapeptide sequence of PRPZPRP (SEQ ID No:5); where Z denotes either R, G, L or W (Table 1). The observed PXP repeats in naturally-occurring PRAMPs were retained in the heptapeptide sequence. Position Z was incorporated to introduce amino acid variability, resulting in four sequence subsets namely PRPRPRP (SEQ ID No:3), PRPGPRP (SEQ ID No:6), PRPLPRP (SEQ ID No:7) and PRPWPRP (SEQ ID No:8) sequences (Table 1). Amino acid variability was integrated in the design to ‘fine-tune’ the overall physicochemical property of SPRLP at the heptapeptide portion. For instance, incorporation of L-arginine imparts an additional protonizable guanidine side-chain whereas L-leucine imparts additional hydrophobicity. L-tryptophan was added for its aromatic ring side-chain while L-glycine was selected to see the effect of replacing the carbon-based side-chain groups into hydrogen. Aliphatic lipids such as octanoic acid (C8), dodecanoic acid (C12) and hexadecanoic acid (C16) were ligated to the N-terminus of the cationic heptapeptide to vary the hydrophobic or amphiphilic moment in the SPRLPs (24-26). We also included the more rigid and conformationally-constrained lipid 1-adamantaneacetic acid (Ad) in our study. The bulky hydrophobic adamantane moiety is perceived to be less prone to toxicity issues relative to longer aliphatic hydrocarbons. The C-terminus of each of the peptides were also amidated. Sixteen acylated SPRLPs were synthesized to explore the effect of peptide sequence and amphiphilicity to their biological activity.

### EXAMPLE 3—SPRLPs COMPOSED OF LONGER HYDROCARBONS DEMONSTRATE ANTIBACTERIAL ACTIVITY

The synthesized SPRLPs were evaluated for their antibacterial potency against a panel of Gram-positive and Gram-negative bacteria (Tables 2 & 3). Some of the included pathogens were collected from patients visiting or admitted to participating Canadian hospitals through the CAN-ICU (27) and CANWARD (28) national surveillance studies. Antibacterial activity was assessed using minimum inhibitory concentration (MIC) against various clinical pathogens.

The Gram-negative-specific antibacterial activity of naturally-existing PRAMPs was not observed for the synthesized SPRLPs. Among the four sequence subsets, peptides acylated with C16 displayed better antibacterial activity relative to C8, C12 or Ad. Three out of the four C16-comprising peptides showed promising activity. C16-PRP displayed broad-spectrum activity (Table 2) with an MIC range of 4-16 μg/mL against Gram-positive bacteria and an MIC range of 8-16 μg/mL against the Gram-negative bacteria E. coli. Moderate activity against Gram-positive bacteria (MIC range of 8-32 μg/mL) was demonstrated by C16-PGP (Table 2). The lipopeptide C16-PWP also exhibited good activity (MIC range of 4-8 μg/mL) against Gram-positive bacteria (Table 3). Overall, these SPRLPs reported herein appeared to be mostly active against Gram-positive organisms.

### EXAMPLE 4—NON-SPECIFIC MEMBRANE LYSIS LIMITS THERAPEUTIC POTENTIAL

Since PRAMPs are able to kill bacteria through membrane lysis, it is imperative to evaluate whether these synthesized SPRLPs also lyse eukaryotic membranes. The ability to lyse porcine red blood cells was assessed and the minimum concentration to result in 5% erythrocyte hemolysis (MHC) was reported (Tables 2 & 3). SPRLPs acylated with the long hydrocarbon C16 showed high hemolytic activity. C16-PRP, C16-PGP, C16-PLP and C16-PWP resulted in 5% red blood cell hemolysis at 16 μg/mL. This data corroborates that the observed antibacterial activity of these four SPRLPs is through non-specific membrane lysis. This greatly limits their therapeutic potential. The lipopeptide C12-PWP demonstrated marginal hemolytic activity with a MHC of 64 μg/mL. However, all other SPRLPs were non-hemolytic (MHC>512 μg/mL).

### EXAMPLE 5—POTENTIATION OF MINOCYCLINE AND RIFAMPICIN BY AN SPRLP AGAINST MDR/XDR P. aeruginosa

Adjuvants typically do not kill the pathogens directly but are able to help their antibiotic partner broaden their antibacterial spectrum or maximize antibacterial activity. Literature search revealed only one report of PRAMP that can synergize a clinically-used antibiotic against Gram-negative bacilli. The long proline-rich peptide dimer A3-APO, consisting of 41 amino acids, was found to potentiate chloramphenicol against Klebsiella pneumoniae using a checkerboard assay (29). However, an amphiphilic lysine-based peptide-like agent was reported to potentiate rifampicin in E. coli (30). We therefore were interested to study whether our short proline-rich heptapeptide-based SPRLPs possessed adjuvant properties. Certainly, it is advantageous to have a lead molecule of shorter peptide sequence as it is more cost-effective and more pliable to peptidomimetic modifications for further optimization.

### MATERIALS AND METHODS

### Peptide Preparation

All lipopeptides were synthesized on solid-phase methylbenzhydrylamine (MBHA) Rink amide resin following standard fluorenylmethyloxycarbonyl (Fmoc) chemistry protocol (37, 38). Amino acids with reactive side-chain functional group were masked with protecting groups inert to solid-phase peptide synthesis conditions yet labile upon peptide cleavage from the solid support. Therefore, Fmoc-Arg(Pbf)-OH and Fmoc-Trp(Boc)-OH were purchased to prevent the guanidine and indole side-chain, respectively, from causing unwanted reaction. The coupling reagent O-(benzotriazol-1-yl)-N,N,N′,N′-tetramethyluronium tetrafluoroborate (TBTU) and N-methylmorpholine were used to induce peptide bond formation between amino acids. All reagents and solvents were purchased from commercially available sources and used without further purification.

SPRLPs were purified via reverse-phase flash chromatography using C18 (40-63 μm) silica gel purchased from Silicycle (USA). Purity was assessed by high-performance liquid chromatography (HPLC) and were determined to be >95%. Each peptide was characterized using nuclear magnetic resonance (NMR) and mass spectrometry (MS). One (1H and 13C) and two-dimensional NMR experiments were performed on either a Bruker AMX-500 or AMX-300 (Germany). Electrospray ionization mass spectrometry (ESI-MS) experiments were performed on Varian 500-MS ion trap mass spectrometer (USA) and high-resolution matrix-assisted laser desorption ionization mass spectrometry (MALDI-MS) experiments were done on Bruker Ultraflextreme mass spectrometer (Germany) coupled to a time-of-flight mass analyzer.

### Bacterial Strains

Isolates used in this study were either obtained from the American Type Culture Collection (ATCC), the Canadian National Intensive Care Unit (CAN-ICU) surveillance study (27) or the Canadian Ward Surveillance (CANWARD) study (28). Clinical isolates belonging to the CAN-ICU and CANWARD studies were recovered from patients suffering a presumed infectious disease entering or admitted in a participating medical center across Canada during the time of study. MDR P. aeruginosa strains in this study refer to those that are resistant to aminoglycosides, fluoroquinolones, cephalosporins, and carbapenems while XDR strains are those that are resistant to aminoglycosides, fluoroquinolones, cephalosporins, carbapenems, aztreonam and penicillin+β-lactamase inhibitor combination (39, 40).

### Antimicrobial Susceptibility Assay

Microbroth dilution susceptibility test following the Clinical and Laboratory Standards Institute (CLSI) guidelines (32) was performed to assess the in vitro antibacterial activity of SPRLPs. Overnight grown bacterial cultures were diluted in saline to achieve a 0.5 McFarland turbidity, followed by 1:50 dilution in Mueller-Hinton broth (MHB) for inoculation to a final concentration of 5×10⁵ colony forming units/mL. The assay was done on a 96-well plate to which the agents of interest were 2-fold serially diluted in MHB and incubated with equal volumes of inoculum at 37° C. for 18 hours. MIC was determined as the lowest concentration to inhibit visible bacterial growth in form of turbidity, which was confirmed using EMax Plus microplate reader (Molecular Devices, USA) at a wavelength of 590 nm. The well containing MHB broth with or without bacterial cells was used as positive or negative control, respectively.

### Hemolytic Assay

The ability of SPRLPs to lyse eukaryotic red blood cells were quantified by the amount of hemoglobin released upon incubation with pig erythrocytes, following published protocols (37, 39). Fresh pig blood drawn from pig antecubital vein was centrifuged at 1000×g for 5 minutes at 4° C., washed with PBS three times and re-suspended in the same buffer, consecutively. Then, agents of interest were 2-fold serially diluted in PBS on 96-well plate and mixed with equal volumes of erythrocyte solution. Post 1-hour incubation at 37° C., intact cells were pelleted by centrifugation at 1000×g for 5 minutes at 4° C. The supernatant was then transferred to a new 96-well plate. The hemoglobin released was measured via EMax Plus microplate reader (Molecular Devices, USA) at 570 nm wavelength. Erythrocytes in PBS with or without 0.1% Triton X-100 was used as negative or positive control, respectively.

### Synergy Scan Testing

The assay was performed on a 96-well plate, to which the agents of interest was 2-fold serially diluted in working MHB. Prior to serial dilution, SPRLP was added to the working MHB media so that a fixed final concentration of 8 μg/mL (5 μM) SPRLP per well was achieved. To ensure that the assay was working, the MIC determination test of the studied antibiotic (without SPRLP) was included on the same plate. Similar MIC results between the assay comparator and an independent MIC determination test (on a different plate) ensured validity of the synergy test. Overnight grown bacterial cultures were diluted in saline to 0.5 McFarland turbidity, followed by 1:50 dilution in MHB (without SPRLP) and inoculation into each well to a final concentration of approximately 5×10⁵ colony forming units/mL. Wells containing only MHB (without SPRLP) with or without bacterial cells were used as positive or negative control, respectively. The plate was then incubated at 37° C. for 18 hours and examined for visible turbidity, to which was confirmed using EMax Plus microplate reader (Molecular Devices, USA) at a wavelength of 590 nm. A 4-fold or more MIC reduction of antibiotic in the presence of 8 μg/mL (5 μM) SPRLP denoted a positive synergy result and was further validated by a checkerboard assay.

### Checkerboard Assay

The assay was done on a 96-well plate as previously described (40, 41). The agent of interest was 2-fold serially diluted along the x-axis, while the adjuvant was 2-fold serially diluted along the y-axis to create a matrix in which each well consist of a combination of both at different concentrations. Overnight grown bacterial cultures were diluted in saline to 0.5 McFarland turbidity, followed by 1:50 dilution in MHB and inoculation on each well to a final concentration of approximately 5×10⁵ colony forming units/mL. Wells containing only MHB with or without bacterial cells were used as positive or negative control, respectively. The plate was incubated at 37° C. for 18 hours and examined for visible turbidity, to which was confirmed using EMax Plus microplate reader (Molecular Devices, USA) at a wavelength of 590 nm. Fractional inhibitory concentration (FIC) of antibiotic was calculated by dividing the MIC of antibiotic in the presence of adjuvant by the MIC of antibiotic alone. Similarly, the FIC of adjuvant was calculated via dividing the MIC of adjuvant in the presence of antibiotic by the MIC of adjuvant alone. FIC index was obtained by the summation of both FIC values.

FIC index was interpreted as synergistic, indifferent or antagonistic for values of ≤0.5, 0.5<x≤4 or >4, respectively (31).

### Proliferation Assay

The CyQuant Direct cell proliferation assay kit (ThermoFisher, Canada) was used to assess the effect of C12-PRP on cell proliferation according to the manufacturer's protocol. Briefly, human embryonic kidney cells (HEK-293) and HepG2 cells were grown in Dulbecco's Modified Eagle's Medium supplemented with 10% fetal bovine serum. The cells were dispersed into 96-well plates (8000 cells/well in 100 μl ). Wells with media but no cells were used as blanks. After 24 hours, varying concentrations of C12-PRP, colistin and Adriamycin® were added to the wells containing cells but also the blanks. After incubation of the cells with the compounds for 48 hours, the CyQuant Direct detection reagent was added to the wells. Plates were incubated at 37° C. for 1 hour and the fluorescence (Excitation 480nm/Emission 535 nm) was read using a SpectraMax M2e (Molecular Devices, USA). As a positive control, CyQuant Direct detection reagent was added to a plate with untreated cells, incubated for 1 hour followed by fluorescence reading. The number of cells in each well was determined by detaching the cells by trypsin followed by counting on a CoulterZM counter to ensure an approximate equal number of cells per well.

### Cytotoxicity Assay

The cytotoxic effects of C12-PRP was assessed by measuring its effect on the viability of HEK-293 or HepG2 cells. The cells were dispersed into 96-well plates and after 24 hours, C12-PRP, colistin or Adriamycin® were added as described in section 2.7. After incubation for 48 hours, the viability of the cells was determined with the MTS reagent (Promega, Canada) as previously described (42).

### Statistical Analysis

Data herein represents the mean±standard deviation (error bars) of at least three independent experiments. The null hypothesis was evaluated via one-way analysis of variance (ANOVA), where the confidence interval was set to be 95% (*p<0.05).

While the preferred embodiments of the invention have been described above, it will be recognized and understood that various modifications may be made therein, and the appended claims are intended to cover all such modifications which may fall within the spirit and scope of the invention.

### REFERENCES

1. Safaei H G, Moghim S, Isfahani B N, Fazeli H, Poursina F, Yadegari S, Nasirmoghadas P, Hosseininassab Nodoushan S A. 2017. Distribution of the strains of multidrug-resistant, extensively drug-resistant, and pandrug-resistant Pseudomonas aeruginosa isolates from burn patients. Adv Biomed Res 6:74. doi:10.4103/abr.abr_239_16.

2. Cerceo E, Deitelzweig S B, Sherman B M, Amin A N. 2016. Multidrug-resistant Gram-negative bacterial infections in the hospital setting: Overview, implications for clinical practice, and emerging treatment options. Microb Drug Resist 22:412-431. doi:10.1089/mdr.2015.0220.

3. Potron A, Poirel L, Nordmann P. 2015. Emerging broad-spectrum resistance in Pseudomonas aeruginosa and Acinetobacter baumannii: Mechanisms and epidemiology. Int J Antimicrob Agents 45:568-585. doi:10.1016/j.ijantimicag.2015.03.001.

4. Gellatly S L, Hancock R E W. 2013. Pseudomonas aeruginosa: new insights into pathogenesis and host defenses. Pathog Dis 67:159-173. doi:10.1111/2049-632X.12033.

5. Breidenstein E B M, de la Fuente-Nunez C, Hancock R E W. 2011. Pseudomonas aeruginosa: all roads lead to resistance. Trends Microbiol 19:419-246. doi:10.1016/j.tim.2011.04.005.

6. Kumar A, Schweizer H P. 2005. Bacterial resistance to antibiotics: active efflux and reduced uptake. Adv Drug Deliv Rev 57:1486-1513. doi:10.1016/j.addr.2005.04.004.

7. Silver L L. 2016. A Gestalt approach to Gram-negative entry. Bioorg Med Chem 24:6379-6389. doi:10.1016/j.bmc.2016.06.044.

8. Huwaitat R, McCloskey A P, Gilmore B F, Laverty G. 2016. Potential strategies for the eradication of multidrug-resistant Gram-negative bacterial infections. Future Microbiol 11:955-972. doi:10.2217/fmb-2016-0035.

9. Li W, Tailhades J, O'Brien-Simpson N M, Separovic F, Otvos L J, Hossain M A, Wade J D. 2014. Proline-rich antimicrobial peptides: potential therapeutics against antibiotic-resistant bacteria. Amino Acids 46:2287-2294. doi:10.1007/s00726-014-1820-1.

10. Domalaon R, Zhanel G G, Schweizer F. 2016. Short antimicrobial peptides and peptide scaffolds as promising antibacterial agents. Curr Top Med Chem 16:1217-1230. doi:10.2174/1568026615666150915112459.

11. Benincasa M, Scocchi M, Podda E, Skerlavaj B, Dolzani L, Gennaro R. 2004. Antimicrobial activity of Bac7 fragments against drug-resistant clinical isolates. Peptides 25:2055-2061. doi:10.1016/j.peptides.2004.08.004.

12. Holani R, Shah C, Haji Q, Inglis G D, Uwiera R R E, Cobo E R. 2016. Proline-arginine rich (PR-39) cathelicidin: Structure, expression and functional implication in intestinal health. Comp Immunol Microbiol Infect Dis 49:95-101. doi:10.1016/j.cimid.2016.10.004.

13. Li W-F, Ma G-X, Zhou X-X. 2006. Apidaecin-type peptides: biodiversity, structure-function relationships and mode of action. Peptides 27:2350-2359. doi:10.1016/j.peptides.2006.03.016.

14. Krizsan A, Volke D, Weinert S, Strater N, Knappe D, Hoffmann R. 2014. Insect-derived proline-rich antimicrobial peptides kill bacteria by inhibiting bacterial protein translation at the 70S ribosome. Angew Chem Int Ed Engl 53:12236-12239. doi:10.1002/anie.201407145.

15. Podda E, Benincasa M, Pacor S, Micali F, Mattiuzzo M, Gennaro R, Scocchi M. 2006. Dual mode of action of Bac7, a proline-rich antibacterial peptide. Biochim Biophys Acta 1760:1732-1740. doi:10.1016/j.bbagen.2006.09.006.

16. Holfeld L, Hoffmann R, Knappe D. 2017. Correlating uptake and activity of proline-rich antimicrobial peptides in Escherichia coli. Anal Bioanal Chem 409:5581-5592. doi:10.1007/s00216-017-0496-2.

17. Runti G, Lopez Ruiz M del C, Stoilova T, Hussain R, Jennions M, Choudhury H G, Benincasa M, Gennaro R, Beis K, Scocchi M. 2013. Functional characterization of SbmA, a bacterial inner membrane transporter required for importing the antimicrobial peptide Bac7(1-35). J Bacteriol 195:5343-5351. doi:10.1128/JB.00818-13.

18. Graf M, Mardirossian M, Nguyen F, Seefeldt A C, Guichard G, Scocchi M, Innis C A, Wilson D N. 2017. Proline-rich antimicrobial peptides targeting protein synthesis. Nat Prod Rep 34:702-711. doi:10.1039/c7np00020k.

19. Runti G, Benincasa M, Giuffrida G, Devescovi G, Venturi V, Gennaro R, Scocchi M. 2017. The mechanism of killing by the proline-rich peptide Bac7(1-35) against clinical strains of Pseudomonas aeruginosa differs from that against other Gram-negative bacteria. Antimicrob Agents Chemother 61:e01660-16. doi:10.1128/AAC.01660-16.

20. Gill E E, Franco O L, Hancock R E W. 2015. Antibiotic adjuvants: diverse strategies for controlling drug-resistant pathogens. Chem Biol Drug Des 85:56-78. doi:10.1111/cbdd.12478.

21. Bernal P, Molina-Santiago C, Daddaoua A, Llamas M A. 2013. Antibiotic adjuvants: identification and clinical use. Microb Biotechnol 6:445-449. doi:10.1111/1751-7915.12044.

22. Toussaint K A, Gallagher J C. 2015. beta-lactam/beta-lactamase inhibitor combinations: from then to now. Ann Pharmacother 49:86-98. doi:10.1177/1060028014556652.

23. Shlaes D M. 2013. New beta-lactam-beta-lactamase inhibitor combinations in clinical development. Ann N Y Acad Sci 1277:105-114. doi:10.1111/nyas.12010.

24. Sivertsen A, Isaksson J, Leiros H-K S, Svenson J, Svendsen J-S, Brandsdal B O. 2014. Synthetic cationic antimicrobial peptides bind with their hydrophobic parts to drug site II of human serum albumin. BMC Struct Biol 14:4. doi:10.1186/1472-6807-14-4.

25. Findlay B, Szelemej P, Zhanel G G, Schweizer F. 2012. Guanidylation and tail effects in cationic antimicrobial lipopeptoids. PLoS One 7:e41141. doi:10.1371/journal.pone.0041141.

26. Svenson J, Brandsdal B-O, Stensen W, Svendsen J S. 2007. Albumin binding of short cationic antimicrobial micropeptides and its influence on the in vitro bactericidal effect. J Med Chem 50:3334-3339. doi:10.1021/jm0703542.

27. Zhanel G G, DeCorby M, Laing N, Weshnoweski B, Vashisht R, Tailor F, Nichol K A, Wierzbowski A, Baudry P J, Karlowsky J A, Lagacé-Wiens P, Walkty A, McCracken M, Mulvey M R, Johnson J, Canadian Antimicrobial Resistance Alliance (CARA), Hoban D J. 2008. Antimicrobial-resistant pathogens in intensive care units in Canada: results of the Canadian National Intensive Care Unit (CAN-ICU) study, 2005-2006. Antimicrob Agents Chemother 52:1430-1437. doi:10.1128/AAC.01538-07.

28. Zhanel G G, Adam H J, Baxter M R, Fuller J, Nichol K a., Denisuik A J, Lagacé-Wiens P, Walkty A, Karlowsky J A, Schweizer F, Hoban D J, Canadian Antimicrobial Resistance Alliance. 2013. Antimicrobial susceptibility of 22746 pathogens from Canadian hospitals: results of the CANWARD 2007-11 study. J Antimicrob Chemother 68:Suppl 1:7-22. doi:10.1093/jac/dkt022.

29. Cassone M, Vogiatzi P, La Montagna R, De Olivier Inacio V, Cudic P, Wade J D, Otvos L Jr. 2008. Scope and limitations of the designer proline-rich antibacterial peptide dimer, A3-APO, alone or in synergy with conventional antibiotics. Peptides 29:1878-1886. doi:10.1016/j.peptides.2008.07.016.

30. Jammal J, Zaknoon F, Kaneti G, Goldberg K, Mor A. 2015. Sensitization of Gram-negative bacteria to rifampin and OAK combinations. Sci Rep 5:9216. doi:10.1038/srep09216.

31. Meletiadis J, Poumaras S, Roilides E, Walsh T J. 2010. Defining fractional inhibitory concentration index cutoffs for additive interactions based on self-drug additive combinations, Monte Carlo simulation analysis, and in vitro-in vivo correlation data for antifungal drug combinations against Aspergillus fumi. Antimicrob Agents Chemother 54:602-609. doi:10.1128/AAC.00999-09.

32. The Clinical and Laboratory Standards Institute. Performance Standards for Antimicrobial Susceptibility Testing CLSI supplement M100S 26th ed. Clin Lab Stand Institute, Wayne, Pa. 2016.

33. Qvit N, Rubin S J S, Urban T J, Mochly-Rosen D, Gross E R. 2017. Peptidomimetic therapeutics: scientific approaches and opportunities. Drug Discov Today 22:454-462. doi:10.1016/j.drudis.2016.11.003.

34. Weinstock M T, Francis J N, Redman J S, Kay M S. 2012. Protease-resistant peptide design-empowering nature's fragile warriors against HIV. Biopolymers 98:431-442. doi:10.1002/bip.22073.

35. Srinivas N, Jetter P, Ueberbacher B J, Werneburg M, Zerbe K, Steinmann J, Van der Meijden B, Bernardini F, Lederer A, Dias R L, Misson P E, Henze H, Zumbrunn J, Gombert F O, Obrecht D, Hunziker P, Schauer S, Ziegler U, Käch A, Eberl L, Riedel K, DeMarco S J, Robinson J A. 2010. Peptidomimetic antibiotics target outer-membrane biogenesis in Pseudomonas aeruginosa. Science 327:1010-1013. doi: 10.1126/science.1182749.

36. Mensa B, Howell G L, Scott R, DeGrado W F. 2014. Comparative mechanistic studies of brilacidin, daptomycin, and the antimicrobial peptide LL16. Antimicrob Agents Chemother 58:5136-5145. doi: 10.1128/AAC.02955-14.

37. Domalaon R, Findlay B, Ogunsina M, Arthur G, Schweizer F. 2016. Ultrashort cationic lipopeptides and lipopeptoids: Evaluation and mechanistic insights against epithelial cancer cells. Peptides 84:58-67. doi:10.1016/j.peptides.2016.07.007.

38. Domalaon R, Yang X, O'Neil J, Zhanel G G, Mookherjee N, Schweizer F. 2014. Structure-activity relationships in ultrashort cationic lipopeptides: the effects of amino acid ring constraint on antibacterial activity. Amino Acids 46:2517-2530. doi:10.1007/s00726-014-1806-z.

39. Lyu Y, Yang X, Goswami S, Gorityala B K, Idowu T, Domalaon R, Zhanel G G, Shan A, Schweizer F. 2017. Amphiphilic tobramycin-lysine conjugates sensitize multidrug resistant Gram-negative bacteria to rifampicin and minocycline. J Med Chem 60:3684-3702. doi:10.1021/acs.jmedchem.6b01742.

40. Yang X, Goswami S, Gorityala B K, Domalaon R, Lyu Y, Kumar A, Zhanel G G, Schweizer F. 2017. A tobramycin vector enhances synergy and efficacy of efflux pump inhibitors against multidrug-resistant Gram-negative bacteria. J Med Chem 60:3913-3932. doi:10.1021/acs.jmedchem.7b00156.

41. Domalaon R, Yang X, Lyu Y, Zhanel G G, Schweizer F. 2017. Polymyxin B3-tobramycin hybrids with Pseudomonas aeruginosa-selective antibacterial activity and strong potentiation of rifampicin, minocycline, and vancomycin. ACS Infect Dis doi: 10.1021/acsinfecdis.7b00145.

42. Ogunsina M, Samadder P, Idowu T, Arthur G, Schweizer F. 2017. Replacing D-glucosamine with its L-enantiomer in glycosylated antitumor ether lipids (GAELs) retains cytotoxic effect against epithelial cancer cells and cancer stem cells. J Med Chem 60:2142-2147. doi:10.1021/acs.jmedchem.6b01773.

43. El Zowalaty M. E, Al Thani A. A, Webster T. J, El Zowalaty A. E, Schweizer H. P, Nasrallah G. K, Marei H. E, Ashour H. M. Pseudomonas aeruginosa: arsenal of resistance mechanisms, decades of changing resistance profiles, and future antimicrobial therapies. Future Microbiol. 2015;10(10):1683-706.

44. Fujii A, Seki M, Higashiguchi M, Tachibana I, Kumanogoh A, Tomono K. Community-acquired, hospital-acquired, and healthcare-associated pneumonia caused by Pseudomonas aeruginosa. Respir Med Case Rep. 2014; 12:30-3.

45. Sligl W I, Dragan T, Smith S W. Nosocomial Gram-negative bacteremia in intensive care: epidemiology, antimicrobial susceptibilities, and outcomes. Int J Infect Dis. 2015; 37: 129-34.

46. Mica Paul and Leonard Leibovici, Combination therapy for Pseudomonas aeruginosa bacteremia: where do we stand? Clinical Infectious Diseases, 2013, 57, 217-220.

47. Pena C, Suarez C, Tubau F, Dominguez A, Sora M, Pujol M, Gudiol F, Ariza J. Carbapenem-resistant Pseudomonas aeruginosa: factors influencing multidrug-resistant acquisition in non-critically ill patients. Eur J Clin Microbiol Infect Dis. 2009; 28(5):519-22.

48. Garnacho-Montero J, Sa-Borges M, Sole-Violan J, Barcenilla F, Escoresca-Ortega A, Ochoa M, Cayuela A, Rello J. Optimal management therapy for Pseudomonas aeruginosa ventilator-associated pneumonia: an observational, multicenter study comparing monotherapy with combination antibiotic therapy. Crit Care Med. 2007; 35(8):1888-95

49. Rahal J. J. Novel antibiotic combinations against infections with almost completely resistant Pseudomonas aeruginosa and Acinetobacter species. Clin Infect Dis 2006; 43 Suppl 2: S95.

50. Kmeid J. G, Youssef M. M, Kanafani Z. A, Kanj S. S. Combination therapy for Gram-negative bacteria: what is the evidence? Expert Rev Anti Infect Ther 2013; 11:1355.

51. Dubois V, Arpin C, Melon M, et al. Nosocomial outbreak due to a multi resistant strain of Pseudomonas aeruginosa P12: efficacy of cefepime-amikacin therapy and analysis of beta-lactam resistance. J Clin Microbiol 2001; 39:2072.

52. Jureti\acute{ }c, D., Vuki\check{ }cevi\acute{ }c, D., Petrov, D., Novkovi\acute{ }c, M., Bojovi\acute{ }c, V., Lu\check{ }ci\acute{ }c, B., Ili\acute{ }c, N., Tossi, A.: Knowledge-based computational methods for identifying or designing novel, non-homologous antimicrobial peptides. Euro. Biophys. J. 40(4), 371-385 (2011).

53. Strøm, M. B., B. E. Haug, M. L. Skar, W. Stensen, T. Stiberg, and J. S. Svendsen. 2003. The pharmacophore of short cationic antibacterial peptides. J. Med. Chem. 46:1567-1570.

54. Zasloff M. Antimicrobial peptides of multicellular organisms. 2002, Nature 415, 389-395

55. Matsuzaki K. Why and how are peptide-lipid interactions utilized for self-defense? Magainins and tachyplesins as archetypes. 1999, Biochim Biophys Acta 1462, 1-10

56. Berthold N, Hoffmann R. Cellular uptake of apidaecin 1b and related analogs in Gram-negative bacteria reveals novel antibacterial mechanism for proline-rich antimicrobial peptides. 2014, Protein Pept Lett. 21, 391-398

TABLE 1
SPRLPs sequences under consideration

| | | Molecular |
|---|---|---|
| | | weight, g/mol |
| Compound | Sequence | (TFA salt) |
| C8-PRP | CH₃(CH₂)₆CO-PRPRPRP^{a}-NH₂ | 1342.33 |
| C12-PRP | CH₃(CH₂)₁₀CO-PRPRPRP^{a}-NH₂ | 1398.44 |
| C16-PRP | CH₃(CH₂)₁₄CO-PRPRPRP^{a}-NH₂ | 1454.55 |
| Ad-PRP | Adamantyl-CH₂CO-PRPRPRP^{a}-NH₂ | 1392.39 |
| C8-PGP | CH₃(CH₂)₆CO-PRPGPRP^{b}-NH₂ | 1129.17 |
| C12-PGP | CH₃(CH₂)₁₀CO-PRPGPRP^{b}-NH₂ | 1185.28 |
| C16-PGP | CH₃(CH₂)₁₄CO-PRPGPRP^{b}-NH₂ | 1241.39 |
| Ad-PGP | Adamantyl-CH₂CO-PRPGPRP^{b}-NH₂ | 1179.23 |
| C8-PLP | CH₃(CH₂)₆CO-PRPLPRP^{c}-NH₂ | 1185.28 |
| C12-PLP | CH₃(CH₂)₁₀CO-PRPLPRP^{c}-NH₂ | 1241.39 |
| C16-PLP | CH₃(CH₂)₁₄CO-PRPLPRP^{c}-NH₂ | 1297.50 |
| Ad-PLP | Adamantyl-CH₂CO-PRPLPRP^{c}-NH₂ | 1235.34 |
| C8-PWP | CH₃(CH₂)₆CO-PRPWPRP^{d}-NH₂ | 1258.34 |
| C12-PWP | CH₃(CH₂)₁₀CO-PRPWPRP^{d}-NH₂ | 1314.44 |
| C16-PWP | CH₃(CH₂)₁₄CO-PRPWPRP^{d}-NH₂ | 1370.55 |
| Ad-PWP | Adamantyl-CH₂CO-PRPWPRP^{d}-NH₂ | 1308.40 |
| C12-prp | CH₃(CH₂)₁₀CO-prprprp^{e}-NH₂ | 1398.44 |
| | (all D-peptide) | |

a-SEQ ID No: 3; b-SEQ ID No: 6; c-SEQ ID No: 7; d-SEQ ID No: 8; e-SEQ ID No: 4

TABLE 2
Biological activity of SPRLPs belonging
to PRP and PGP sequence subsets

| | MIC, μg/mL | | | | | | | |
|---|---|---|---|---|---|---|---|---|
| | C8- | C12- | C16- | Ad- | C8- | C12- | C16- | Ad- |
| Organism | PRP | PRP | PRP | PRP | PGP | PGP | PGP | PGP |
| S. aureus^{a} | >128 | 128 | 8 | >128 | >128 | >128 | 32 | >128 |
| MRSA^{b} | >128 | >128 | 16 | >128 | >128 | >128 | 32 | >128 |
| MSSE^{c} | >128 | 32 | 4 | >128 | >128 | 128 | 8 | >128 |
| MRSE^{d} | >128 | 128 | 8 | >128 | >128 | >128 | 16 | >128 |
| E. faecalis^{e} | >128 | >128 | 16 | >128 | >128 | >128 | 16 | >128 |
| E. faecium^{f} | >128 | 128 | 8 | >128 | >128 | >128 | 16 | >128 |
| E. coli^{g} | >128 | >128 | 16 | >128 | >128 | >128 | 32 | >128 |
| E. coli^{h} | >128 | >128 | 8 | >128 | >128 | >128 | 32 | >128 |
| E. coliⁱ | >128 | >128 | 8 | >128 | >128 | >128 | 32 | >128 |
| E. coli^{j} | >128 | >128 | 8 | >128 | >128 | >128 | 32 | >128 |
| P. aeruginosa^{k} | >128 | >128 | 32 | >128 | >128 | >128 | 128 | >128 |
| P. aeruginosa^{l} | >128 | >128 | 32 | >128 | >128 | >128 | 128 | >128 |
| P. aeruginosa^{m} | >128 | >128 | 64 | >128 | >128 | >128 | 128 | >128 |
| P. aeruginosaⁿ | >512 | 128 | 32 | >512 | >512 | >512 | 64 | >512 |
| S. maltophilia^{o} | >128 | >128 | 64 | >128 | >128 | >128 | 128 | >128 |
| A. baumannii^{p} | >128 | >128 | 16 | >128 | >128 | >128 | 32 | >128 |
| K. | >128 | >128 | 64 | >128 | >128 | >128 | 64 | >128 |
| pneumoniae^{q} | | | | | | | | |
| MHC^{r} | >512 | >512 | 16 | >512 | >512 | >512 | 16 | >512 |

^{a}= ATCC 29213.
^{b}= methicillin-resistant S. aureus ATCC 33592.
^{c}= methicillin-susceptible Staphylococcus epidermidis CANWARD-2008 81388.
^{d}= methicillin-resistant S. Epidermidis CAN-ICU 61589 (ceftazidime-resistant).
^{e}= ATCC 29212.
^{f}= ATCC 27270.
^{g}= ATCC 25922.
^{h}= CAN-ICU 61714 (gentamicin-resistant)
ⁱ= CAN-ICU 63074 (amikacin-intermediate resistant).
^{j}= CANWARD-2011 97615 (gentamicin-, tobramycin-, ciprofloxacin-resistant) aac(3′)iia.
^{k}= ATCC 27853.
^{l}= CAN-ICU 62308 (gentamicin-resistant).
^{m}= CANWARD-2011 96846 (gentamicin-, tobramycin-resistant).
ⁿ= wild-type PAO1
^{o}= CAN- ICU 62584.
^{p}= CAN-ICU 63169.
^{q}= ATCC 13883.
^{r}= minimum concentration in μg/mL that resulted in 5% red blood cell hemolysis.

TABLE 3
Biological activity of SPRLPs belonging
to PLP and PWP sequence subsets

| | MIC, μg/mL | | | | | | | |
|---|---|---|---|---|---|---|---|---|
| | C8- | C12- | C16- | Ad- | C8- | C12- | C16- | Ad- |
| Organism | PLP | PLP | PLP | PLP | PWP | PWP | PWP | PWP |
| S. aureus^{a} | >128 | 128 | 64 | >128 | >128 | 32 | 8 | >128 |
| MRSA^{b} | >128 | 128 | 64 | >128 | >128 | 32 | 8 | >128 |
| MSSE^{c} | >128 | 64 | 32 | >128 | >128 | 16 | 4 | >128 |
| MRSE^{d} | >128 | 64 | 64 | >128 | >128 | 16 | 8 | >128 |
| E. faecalis^{e} | >128 | 128 | 64 | >128 | >128 | 32 | 8 | >128 |
| E. faecium^{f} | >128 | 128 | 64 | >128 | >128 | 32 | 8 | >128 |
| E. coli^{g} | >128 | >128 | 128 | >128 | >128 | 128 | 32 | >128 |
| E. coli^{h} | >128 | >128 | 64 | >128 | >128 | 128 | 32 | >128 |
| E. coliⁱ | >128 | >128 | 64 | >128 | >128 | 128 | 16 | >128 |
| E. coli^{j} | >128 | >128 | 128 | >128 | >128 | 128 | 64 | >128 |
| P. aeruginosa^{k} | >128 | >128 | >128 | >128 | >128 | >128 | 64 | >128 |
| P. aeruginosa^{l} | >128 | >128 | >128 | >128 | >128 | >128 | 64 | >128 |
| P. aeruginosa^{m} | >128 | >128 | >128 | >128 | >128 | >128 | 64 | >128 |
| P. aeruginosaⁿ | >512 | 512 | 32 | >512 | >512 | 64 | 32 | >512 |
| S. maltophilia^{o} | >128 | >128 | >128 | >128 | >128 | >128 | 64 | >128 |
| A. baumannii^{p} | >128 | >128 | 128 | >128 | >128 | 128 | 64 | >128 |
| K. | >128 | >128 | 128 | >128 | >128 | >128 | 64 | >128 |
| pneumoniae^{q} | | | | | | | | |
| MHC^{r} | >512 | >512 | 16 | >512 | >512 | 64 | 16 | >512 |

^{a}= ATCC 29213.
^{b}= methicillin-resistant S. aureus ATCC 33592.
^{c}= methicillin-susceptible Staphylococcus epidermidis CANWARD-2008 81388.
^{d}= methicillin-resistant S. epidermidis CAN-ICU 61589 (ceftazidime-resistant).
^{e}= ATCC 29212.
^{f}= ATCC 27270.
^{g}= ATCC 25922.
^{h}= CAN-ICU 61714 (gentamicin-resistant).
ⁱ= CAN-ICU 63074 (amikacin-intermediate resistant).
^{j}= CANWARD-2011 97615 (gentamicin-, tobramycin-, ciprofloxacin-resistant) aac(3′)iia.
^{k}= ATCC 27853.
^{l}= CAN-ICU 62308 (gentamicin-resistant).
^{m}= CANWARD-2011 96846 (gentamicin-, tobramycin-resistant).
ⁿ= wild-type PAO1
^{o}= CAN-ICU 62584.
^{p}= CAN-ICU 63169.
^{q}= ATCC 13883.
^{r}= minimum concentration in μg/mL that resulted in 5% red blood cell hemolysis.

TABLE 4
Adjuvant potency of amphiphilic C12-PRP in combination with
minocycline (MIN) against wild-type and MDR/XDR P. aeruginosa.

| P. | | | | Absolute | |
|---|---|---|---|---|---|
| aeruginosa | MIC_{MIN}, | MIC_{C12-PRP}, | | MIC_{MIN},^{a} | Poten- |
| strain | μg/mL | μg/mL | FIC index | μg/mL | tiation^{b} |
| PAO1 | 8 | 128 | 0.19 | 1 | 8-fold |
| 259-96918 | 16 | >128 | 0.12 < x < | 2 | 8-fold |
| | | | 0.19 | | |
| 260-97103 | 16 | 128 | 0.12 | 1 | 16-fold |
| 262-101856 | 64 | >128 | 0.12 < x < | 16 | 4-fold |
| | | | 0.25 | | |
| 264-104354 | 32 | >128 | 0.06 < x < | 2 | 16-fold |
| | | | 0.12 | | |
| 91433^{c} | 32 | >128 | 0.12 < x < | 8 | 4-fold |
| | | | 0.25 | | |
| 100036 | 16 | >128 | 0.12 < x < | 4 | 4-fold |
| | | | 0.25 | | |
| 101243^{c} | 2 | 128 | 0.31 | 0.5 | 4-fold |
| 101885 | 16 | 64 | 0.37 | 4 | 4-fold |

^{a}= MIC of minocycline in the presence of 8 μg/mL (5 μM) C12-PRP.
^{b}= degree of antibiotic potentiation in the presence of 8 μg/mL (5 μM) C12-PRP.
^{c}= colistin-resistant.
MDR = multidrug-resistant.
XDR = extensively drug-resistant.

TABLE 5
Adjuvant potency of amphiphilic C12-PRP in combination with
rifampicin (RMP) against wild-type and MDR/XDR P. aeruginosa.

| P. | | | | Absolute | |
|---|---|---|---|---|---|
| aeruginosa | MIC_{RMP}, | MIC_{C12-PRP}, | | MIC_{RMP},^{a} | Poten- |
| strain | μg/mL | μg/mL | FIC index | μg/mL | tiation^{b} |
| PAO1 | 8 | 128 | 0.14 | 1 | 8-fold |
| 259-96918 | 16 | >128 | 0.01 < x < | 2 | 8-fold |
| | | | 0.14 | | |
| 260-97103 | 16 | 128 | 0.16 | 2 | 8-fold |
| 262-101856 | 512 | >128 | 0.25 < x < | 512 | none |
| | | | 0.37 | | |
| 264-104354 | 8 | >128 | 0.06 < x < | 0.5 | 16-fold |
| | | | 0.12 | | |
| 91433^{c} | 16 | >128 | 0.06 < x < | 1 | 16-fold |
| | | | 0.09 | | |
| 100036 | 16 | >128 | 0.01 < x < | 1 | 16-fold |
| | | | 0.14 | | |
| 101243^{c} | 4 | 128 | 0.09 | 0.125 | 32-fold |
| 101885 | 16 | 64 | 0.25 | 2 | 8-fold |

^{a}= MIC of rifampicin in the presence of 8 μg/mL (5 μM) C12-PRP.
^{b}= degree of antibiotic potentiation in the presence of 8 μg/mL (5 μM) C12-PRP.
^{c}= colistin-resistant.
MDR = multidrug-resistant.
XDR = extensively drug-resistant.

TABLE 6
Adjuvant potency of amphiphilic C12-prp in combination with
minocycline (MIN) against wild-type and MDR/XDR P. aeruginosa.

| P. | | | | Absolute | |
|---|---|---|---|---|---|
| aeruginosa | MIC_{MIN}, | MIC_{C12-prp}, | | MIC_{MIN},^{a} | Poten- |
| strain | μg/mL | μg/mL | FIC index | μg/mL | tiation^{b} |
| PAO1 | 8 | >128 | 0.25 < x < 0.31 | 2 | 4-fold |
| 259-96918 | 16 | >128 | 0.12 < x < 0.25 | 4 | 4-fold |
| 260-97103 | 16 | >128 | 0.12 < x < 0.19 | 2 | 8-fold |
| 262-101856 | 64 | >128 | 0.12 < x < 0.25 | 16 | 4-fold |
| 264-104354 | 32 | >128 | 0.12 < x < 0.19 | 4 | 8-fold |
| 91433^{c} | 32 | >128 | 0.12 < x < 0.19 | 4 | 8-fold |
| 100036 | 16 | >128 | 0.25 < x < 0.37 | 8 | 2-fold |
| 101243^{c} | 4 | >128 | 0.12 < x < 0.37 | 2 | 2-fold |
| 101885 | 16 | >128 | 0.25 < x < 0.37 | 8 | 2-fold |

^{a}= MIC of minocycline in the presence of 8 μg/mL (5 μM) C12-prp.
^{b}= degree of antibiotic potentiation in the presence of 8 μg/mL (5 μM) of all D-lipopeptide C12-prp.
^{c}= colistin-resistant.
MDR = multidrug-resistant.
XDR = extensively drug-resistant.

TABLE 7
Adjuvant potency of amphiphilic C12-prp in combination with
rifampicin (RMP) against wild-type and MDR/XDR P. aeruginosa.

| P. | | | | Absolute | |
|---|---|---|---|---|---|
| aeruginosa | MIC_{RMP}, | MIC_{C12-prp}, | | MIC_{RMP},^{a} | Poten- |
| strain | μg/mL | μg/mL | FIC index | μg/mL | tiation^{b} |
| PAO1 | 8 | >128 | 0.12 < x < 0.19 | 1 | 8-fold |
| 259-96918 | 16 | >128 | 0.03 < x < 0.16 | 2 | 8-fold |
| 260-97103 | 16 | >128 | 0.06 < x < 0.19 | 4 | 4-fold |
| 262-101856 | 512 | >128 | 0.12 < x < 0.25 | 256 | 2-fold |
| 264-104354 | 8 | >128 | 0.12 < x < 0.25 | 2 | 4-fold |
| 91433^{c} | 16 | >128 | 0.12 < x < 0.14 | 2 | 8-fold |
| 100036 | 16 | >128 | 0.06 < x < 0.19 | 4 | 4-fold |
| 101243^{c} | 4 | >128 | 0.06 < x < 0.19 | 0.5 | 8-fold |
| 101885 | 16 | >128 | 0.12 < x < 0.25 | 4 | 4-fold |

^{a}= MIC of rifampicin in the presence of 8 μg/mL (5 μM) C12-prp.
^{b}= degree of antibiotic potentiation in the presence of 8 μg/mL (5 μM) of all D-lipopeptide C12-prp.
^{c}= colistin-resistant.
MDR = multidrug-resistant.
XDR = extensively drug-resistant.

1. A compound comprising the peptide as set forth in SEQ ID No:3 or SEQ ID No:4 connected to a C9-C13 aliphatic chain.

2. The compound according to claim 1 wherein the peptide is connected to the C9-C13 aliphatic chain at the N-terminus of the peptide.

3. The compound according to claim 1 wherein the peptide is connected to the C9-C13 aliphatic chain via acylation.

4. The compound according to claim 1 wherein the C9-C13 aliphatic chain is a C12 aliphatic chain.

5. The compound according to claim 4 wherein the C12 aliphatic chain is dodecanoic acid.

6. A method of treating a bacterial infection comprising:
coadministering an effective amount of a compound according to any one of claims 1-5 and an effective amount of a suitable antibiotic to an individual in need of such treatment.

7. The method according to claim 6 wherein the individual in need of such treatment is an individual who is suspected of having a bacterial infection or who has been diagnosed with a gram negative bacteria infection.

8. The method according to claim 7 wherein the gram negative bacteria infection is suspected of being caused by or known to be caused by a drug-resistant bacterial strain.

9. A method of permeabilizing a gram negative bacterial membrane comprising:
administering an effective amount of the compound of claim 1 to a suitable gram negative bacterium.

10. (canceled)

11. (canceled)
